SEQUENCE LISTING (10 sequences)

SEQ ID NO: 1 - DNA, 54 bp, Schizosaccharomyces pombe
  sig_peptide (1)..(54)
atgttaatgaaacaaaccttcttgtactttttgctcacttgcgtcgtatccgct

SEQ ID NO: 2 - protein, 18 aa, Schizosaccharomyces pombe
  SIGNAL (1)..(18)
MLMKQTFLYFLLTCVVSA

SEQ ID NO: 3 - DNA, 92 bp, Artificial sequence
  Synthetic PCR primer
taactcgagaccatgttaatgaaacaaaccttcttgtactttttgctcacttgcgtcgtatccgccatggctagcaaaggagaagaactctt

SEQ ID NO: 4 - DNA, 55 bp, Artificial sequence
  Synthetic PCR primer
taagtcgacgcggccgcgctagcggatcctcaatcgatgttgtacagttcatcca

SEQ ID NO: 5 - DNA, 31 bp, Artificial sequence
  Synthetic DNA primer
tctcgagaacatgaagatcaccgctgtcatt

SEQ ID NO: 6 - DNA, 42 bp, Artificial sequence
  Synthetic PCR primer
tccatggcacgcttcttaaggctaactgaaaccacaccagga

SEQ ID NO: 7 - DNA, 31 bp, Artificial sequence
  Synthitic PCR primer
taaccatggctagcaaaggagaagaactctt

SEQ ID NO: 8 - DNA, 55 bp, Artificial sequence; identical to SEQ ID NO: 4
  Synthetic DNA primer

SEQ ID NO: 9 - DNA, 25 bp, Artificial sequence
  Synthetic PCR primer
gtccataaagcttttcaattcatct

SEQ ID NO: 10 - DNA, 31 bp, Artificial sequence
  Synthetic DNA primer
tctgcagagctttttctttccaatttttttt

(19) United States
(12) Patent Application Publication
(10) Pub. No.: US 2008/0227154 A1
(43) Pub. Date: Sep. 18, 2008

(54) Protein Expression

(76) Inventor: Soren Kjaerulff, Hillerod (DK)

Correspondence Address: BIRCH STEWART KOLASCH & BIRCH, PO BOX 747, FALLS CHURCH, VA, 22040-0747, US

(21) Appl. No.: 11/997,120

(22) PCT Filed: Jul. 29, 2005
(86) PCT No.: PCT/DK2005/000513

§ 371 Date: Mar. 25, 2008

(51) Int. Cl. C12P 21/04; C07H 21/04; C12N 15/00; C12N 5/00; C12N 5/02; C12N 1/00

(52) U.S. Cl. 435/69.1; 536/22.1; 435/320.1; 435/325; 435/419; 435/254.11; 435/348

(57) ABSTRACT

Disclosed is chimeric polynucleotides adopted for providing high-yield expression and secretion of recombinant polypeptides. The chimeric polynucleotide of the invention includes a functional secretion signal peptide derived from S. pombe carboxypeptidase Y (CPY). The invention also discloses methods for use of this cpy leader sequence.

### FIELD OF THE INVENTION

The present invention relates to the field of genetic engineering and molecular biology. In particular, the present invention relates to a novel chimeric polynucleotide and its use as a tool for improved protein expression in host cells, notably in Schizosaccharomyces pombe. Furthermore, the present invention relates to vectors containing the polynucleotide and also the use of these in recombinant expression. The invention is particularly relevant in the field of protein expression where the expression product is secreted from recombinant host cells, especially if these host cells are yeast cells. Finally, the invention also pertains to the use of a signal sequence derived from S. pombe CPY for obtaining increased expression and secretion levels from recombinantly transformed host cells.

### BACKGROUND OF THE INVENTION

Production of recombinant, secretory proteins has been one of the major challenges within the biotechnological industry during the last decade or two. Bacterial cells, in particular Escherichia coli and Bacillus subtilis, have been widely used as host cells for the production of recombinant polypeptides. However, expression in prokaryotes is not effective for all polypeptides, and often it is not possible to reproduce the complicated post-translational modifications of eukaryotic proteins and to reproduce the native, biologically functional conformation. Animal cells, such as CHO cells and S2 Drosophila cells, are likely to produce eukaryotic proteins in the natural sterically folded structures. These cells are, however, more difficult to handle than microorganisms, their culture is costly, and production efficiency is low. Fungal cells, such as yeast cells have proven to be well suited for the production of heterologous eukaryotic proteins. They facilitate post-translational processing of polypeptides, such as folding, phosphorylation and glycosylation, and their cultivation methods are well established and non-expensive.

Among the yeasts, Schizosaccharomyces pombe is considered to be closer related than other yeasts to higher eukaryotes with respect to a variety of properties, such as regulation of cell cycle, transcription, chromosomal organisation and RNA splicing (Kaufer N F et al. 1985, Nature 318: 78-80; Russell P R and Nurse P 1986, Cell 45: 781-782; Jones et al. 1988, Cell 53: 659-67; Brys et al. 1998, DNA Cell Biology 17: 349-358).

Post-translational modifications such as glycosylation, phosphorylation and acetylation of proteins produced in S. pombe are apparently similar to that in mammalian cells (Russell and Nurse 1986 supra; Moreno S et al. 1990, Biochemical Journal 267: 697-702; Chappell T G and Warren G J 1989, Cell Biol. 109: 2693-2702; Giga-Hama et al. 1994, Bio/Technology 12: 400-404). Thus, the use of S. pombe as a host for expression of mammalian proteins is more likely to provide a polypeptide close to its native biologically functional form than the use of many other types of yeast, such as S. cerevisiae.

Secretion of the recombinant protein from the yeast cells is generally advantageous since protein purification is facilitated as the recombinant protein is recovered from the culture supernatant rather from the complex mixture of proteins that results when cells are disrupted to release intracellular proteins. Moreover, secretion also reduces the deleterious (e.g. toxic) effect that intracellular over-expression of a heterologous protein may have on the host cell. Finally, secretory production of a foreign protein, which is naturally exported, is also advantageous in that the product is identical or similar to its naturally occurring counterpart, because the protein enters the secretory pathway in the host cells and undergoes appropriate processing such as formation of disulfide bonds and glycosylation.

Secreted proteins or pro-peptides are generally initially expressed as precursors bearing an N-terminal signal or leader peptide. Signal peptides usually comprise a positively charged N-terminus followed by a hydrophobic core, followed by a cleavage site for a signal peptidase. The signal peptide ensures effective translocation of the expressed product across the membrane of the endoplasmic reticulum (ER). The signal peptide is cleaved off from the rest of the protein by the signal peptidase during translocation. Once it has entered the ER, the protein is transported to the Golgi apparatus, and then follows one of a number of routes in the secretory pathway, depending on the particular protein. The protein may be secreted into the culture medium, retained on the cell surface or routed to cell compartments like vacuoles. Depending on the nature of the protein, a number of modifications take place in the ER and Golgi apparatus. These modifications include glycosylation and proteolytic processing of the protein.

The signal peptide used to direct the secretion of a recombinant protein may be the signal peptide from the protein itself, a heterologous signal peptide or a hybrid of a native and a heterologous signal peptide. A problem encountered with the use of signal peptides heterologous to yeast is typically that the signal peptide does not ensure efficient translocation and/or signal peptidase cleavage.

Only a few signal sequences that effectively function in S. pombe have been reported (Tokunaga et al. 1993, Yeast 9: 379-87; Smerdon et al 1995, Gene 165: 313-318; Braspenning et al. 1998, Biochem. and Biophys. Res. Comm. 245: 166-171; Giga-Hama 1997, in “Foreign Gene Expression in fission yeast” edited by Giga-Hama and Kumagai, Springer publishers) and practically no secretory expression vectors have been developed.

In WO 96/23890, the use of the P-factor pre-pro-peptide to direct recombinant protein secretion in S. pombe is described. However, this system relies on efficient cleavage of the precursor protein by the KRP protease. For at least some proteins, inefficient cleavage will be the case resulting in a heterogeneous protein product.

Tabuchi M et al. 1997, J. Bacteriol. 179(13): 4179-4189 reported experiments where the vacuolar protein sorting of carboxypeptidase Y (CPY) from S. pombe was investigated. The work focused on vacuolar protein sorting of a model protein (S. cerevisiae invertase) that was fused in-frame to various fragments of S. pombe CPY. It was demonstrated that secretion of this model protein could be accomplished when the invertase was fused N-terminally to the first 23 amino acids of pre-pro-CPY, and the CPY signal peptide's amino acid sequence was further predicted on the basis of von Heijne's signal sequence theory to be constituted of the first 18 amino acids of the CPY pre-pro-peptide. Tabuchi et al. did not investigate the efficacy of the CPY signal sequence in terms of expression yields and in fact did not utilize the CPY signal sequence for obtaining purified recombinant protein. Hence, Tabuchi et al. did not establish whether or not the S. pombe signal sequence would be a useful tool for recombinant expression and fermentation.

### OBJECT OF THE INVENTION

It is an object of the present invention to provide more efficient expression and/or secretion in host cells, notably in S. pombe. It is a further object to provide polynucleotides and vectors that facilitate this efficient expression and secretion.

### SUMMARY OF THE INVENTION

As detailed above, there is a definite need for alternative and improved expression systems for producing secreted recombinant proteins, especially in the yeast S. pombe. Further, there is also a need for molecular biology tools that enables efficient secretion of expression products from such cells.

The present inventor has surprisingly found that the signal peptide from S. pombe carboxypeptidase Y having the amino acid sequence set forth in SEQ ID NO: 2 directs secretion of polypeptides with a significantly higher efficiency than hitherto known signal peptides useful in this particular organism, and, based on these findings it is concluded that this signal sequence constitutes an important tool in molecular biology for effecting secretion of proteins in a variety of eukaryotic host cells (especially in view of the fact that the biochemistry and biology of S. pombe is recognized as being very similar to that of higher eukaryotic cells).

Hence, in its broadest and most general scope, the present invention relates to a chimeric polynucleotide that comprises a) a first part encoding a functional secretion signal peptide derived from S. pombe carboxypeptidase Y (CPY), and a second part linked directly to the 3′ end of the first portion, wherein the second part encodes an amino acid sequence which is not naturally associated with an S. pombe carboxypeptidase Y signal peptide and which does not include an amino acid sequence constituted by amino acids 1-5 of the S. pombe carboxypeptidase Y pro-peptide, or b) a nucleotide sequence complementary to the nucleotide sequence defined under a).

The invention also provides for a vector that includes the chimeric polypeptide and a host cell carrying said vector of the invention.

Furthermore, the invention provides for a method for recombinant preparation of a polypeptide, comprising a) transforming a host cell with an expression vector that includes a promoter, a coding sequence operably linked thereto, and, optionally, a terminator, wherein said coding sequence comprises a first part encoding a functional secretion signal peptide derived from S. pombe CPY and a second part encoding the polypeptide, said second part being located C-terminally relative to the functional secretion signal peptide, b) culturing the transformed host cells under conditions that facilitate expression of the coding sequence and translocation of the polypeptide whereby the functional secretion signal peptide is cleaved from its linkage to the polypeptide, and c) recovering and optionally purifying the polypeptide from the culture.

Thus, the invention described herein in general provides for the use, in recombinant preparation and isolation of polypeptides, of a functional secretion signal peptide derived from S. pombe carboxypeptidase Y (CPY).

### LEGENDS TO THE FIGURES

FIG. 1: Structure of the pNmt-cpy-gAFP vector.

The principal elements of the pNmt-cpy-gAFP vector are: I) the promoter and transcriptional terminator derived from the S. pombe nmt1 gene (indicated by a solid box and a thick line, respectively); II) the S. pombe autonomously replicating sequence, ars1 (box hatched horizontally); III) the S. pombe marker gene ura4 (open box); IV) the segment comprising the pUC119 β-lactamase gene and the origin of replication (thin line); V) The signal peptide derived from the S. pombe cpy gene (indicated immediately downstream of the nmt1 promoter segment); VI) the green fluorescent AFP (box hatched vertically) coding sequence linked to the cpy gene.

FIG. 2: Structure of the general-purpose secretory pNmt-cpy vector.

The principal elements of the pNmt-cpy vector are: I) the promoter and transcriptional terminator derived from the S. pombe nmt1 gene (indicated by a solid box and a thick line, respectively); II) the S. pombe autonomously replicating sequence, ars1 (box hatched horizontally); III) the S. pombe marker gene ura4 (open box); IV) the segment comprising the pUC119 β-lactamase gene and the origin of replication (thin line); V) The S. pombe CPY signal peptide encoding sequence (indicated immediately downstream of the nmt1 promoter segment); VI) a polylinker with five unique restriction sites, NcoI, BamHI, NheI, NotI and SalI, for cloning in frame with the CPY secretion signal peptide coding sequence.

FIG. 3: Structure of the pNmt-P3-gAFP vector.

The principal elements of the pNmt-P3-gAFP vector are: I) the promoter and transcriptional terminator derived from the S. pombe nmt1 gene (indicated by a solid box and a thick line, respectively); II) the S. pombe autonomously replicating sequence, ars1 (box hatched horizontally); III) the S. pombe marker gene ura4 (open box); IV) the segment comprising the pUC119 β-lactamase gene and the origin of replication (thin line); V) The P3 signal peptide coding sequence derived from the S. pombe map2 gene (indicated immediately downstream of the nmt1 promoter segment); VI) The green fluorescent AFP coding sequence (box hatched vertically) linked to the P3 signal peptide coding sequence.

FIG. 4: Structure of the pNmt-cpy-ura3 vector.

The principal elements of the pNmt-cpy-ura3 vector are: I) the promoter and transcriptional terminator derived from the S. pombe nmt1 gene (indicated by a solid box and a thick line, respectively); II) the S. pombe autonomously replicating sequence, ars1 (box hatched horizontally); III) the S. cerevisiae marker gene ura3 (open box); IV) the segment comprising the pUC119 β-lactamase gene and the origin of replication (thin line); V) The signal peptide coding sequence derived from the S. pombe cpy gene (indicated immediately downstream of the nmt1 promoter segment).

FIG. 5: Structure of the pNmt-cpy-ura3d vector.

The principal elements of the pNmt-cpy-ura3d vector are: I) the promoter and transcriptional terminator derived from the S. pombe nmt1 gene (indicated by a solid box and a thick line, respectively); II) the S. pombe autonomously replicating sequence, ars1 (box hatched horizontally); III) the S. cerevisiae marker gene ura3 (open box) containing a 176 bp 5′ end deletion; IV) the segment comprising the pUC119 β-lactamase gene and the origin of replication (thin line); V) The signal peptide coding sequence derived from the S. pombe cpy gene (indicated immediately downstream of the nmt1 promoter segment).

FIG. 6: Structure of the pNmt-cpy-stb vector.

The principal elements of the pNmt-cpy-stb vector are: I) the promoter and transcriptional terminator derived from the S. pombe nmt1 gene (indicated by a solid box and a thick line, respectively); II) the S. pombe autonomously replicating sequence, ars1 (box hatched horizontally); III) the S. pombe marker gene ura4 (open box); IV) the segment comprising the pUC119 β-lactamase gene and the origin of replication (thin line); V) the S. pombe stb element (box with grid); VI) The signal peptide coding sequence derived from the S. pombe cpy gene (indicated immediately downstream of the nmt1 promoter segment).

### DETAILED DISCLOSURE OF THE INVENTION

### Definitions

In general, the term “amino acid” as used in the present specification and claims is intended to denote naturally occurring L-amino acids, i.e. the 20 genetic encoded amino acids alanine, valine, leucine, isoleucine, proline, methionine, phenylalanine, tryptophan, glycine, serine, threonine, cysteine, tyrosine, asparagine, glutamine, aspartic acid, glutamic acid, lysine, arginine, and histidine, as well as naturally occurring derivatives thereof, such as desmosine, 4-hydroxyproline, 5-hydroxylysine, γ-carboxyglutamic acid, and 6-N-methyllysine.

A “polypeptide” is in the present context intended to mean both short peptides of from 2 to 10 amino acid residues, oligopeptides of from 11 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups, or can include other post-translational modifications.

A “chimeric polynucleotide” as used herein refers to a nucleotide sequence consisting of a first nucleotide sequence encoding at least a signal peptide and a second nucleotide sequence encoding a (poly)peptide not naturally associated with said signal peptide.

A “targeting sequence” is a peptide sequence that effects a specific localisation of protein comprising that sequence. Examples of targeting sequences include localisation sequences and membrane anchoring sequences, but also binding sequences, selective degradation signalling sequences etc. A detailed review of these types of sequences can be found e.g. in WO 97/27213.

A “signal sequence” or “signal peptide” is targeting sequence constituted by an amino acid sequence which, when operably linked to the amino-terminus of a polypeptide, directs the translocation thereof into the endoplasmic reticulum (ER) in a eukaryotic host cell.

A “heterologous polypeptide” as used herein is a polypeptide which is not normally expressed and secreted by the host cell used to express that particular polypeptide.

The term “subsequence” means any consecutive stretch of at least 3 amino acids or, when relevant, of at least 3 nucleotides, derived directly from a longer reference amino acid sequence or nucleic acid sequence, respectively.

A “cloning vector” means a plasmid DNA which can be used to insert a DNA fragment of interest into a host cell, normally in order to produce multiple copies of the fragment and hence the vector.

“Expression vector” means a plasmid or viral DNA containing necessary regulatory signals for the synthesis of mRNA derived from gene sequences, which can be inserted into the vector. The gene sequences being e.g. a chimeric polynucleotide as defined above.

“Promoter region” means a nucleotide sequence that provides a cell with the regulatory sequences for expression of a coding sequence operably linked thereto. In general, a coding sequence is located 3′ to a promoter sequence. The promoter sequence consists of proximal and more distal upstream elements, the latter elements often referred to as enhancers. Accordingly, an “enhancer” is a DNA sequence, which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters, which cause a gene to be expressed in most cell types, at most times are commonly referred to as “constitutive promoters”.

A “terminator sequence” (or just “terminator”) is a DNA sequence, which is recognized by the expression host to terminate transcription. It is operably linked to the 3′-end of the DNA encoding the polypeptide to be expressed.

A “polyadenylation sequence” is a DNA sequence which when transcribed is recognized by the expression host to add polyadenosine residues to transcribed mRNA. It is operably linked to the 3′-end of the DNA encoding the polypeptide to be expressed.

“Translation initiation region” (TIR) as used herein refers to a region of RNA (or its coding DNA) determining the site and efficiency of initiation of translation of a gene of interest.

A “selectable marker” is a genetic element present in an expression vector, which, when expressed, provides an indication of successful transformation of the host cell. For instance, the selectable marker may provide the transformed host cell with resistance to an antibiotic (a dominant type marker) one or with the ability to metabolise a particular nutrient (an auxotrophic type of selectable marker, i.e. a marker that “cures” a deficiency in the host). Typically, the selectable marker is under the control of a promoter that is separate from the promoter that controls expression of the gene to be expressed by the vector.

The term “operably linked” refers to the association of nucleic acid sequences on a single nucleic acid fragment so that the function of one is affected by the other. For example, a promoter is operably linked to a coding sequence when it is capable of affecting the expression of that coding sequence (i.e., that the coding sequence is under the transcriptional control of the promoter).

The term “expression”, as used herein, refers to complete biological process in a host cell that sets out from the transcription and stable accumulation of mRNA derived from the chimeric polynucleotide of the invention through subsequent translation of mRNA into a polypeptide product and finally to post-translational modifications of the polypeptide product effected by the host cell. “Overexpression” refers to the production of a gene product in transformed cells that exceeds levels of production in normal, non-transformed cells.

“S. pombe Carboxypeptidase Y” is the protein having the amino acid sequence CAB10121 (NCBI data base) that is natively encoded by the corresponding nucleotide sequence D86560 (NCBI data base).

A “functional secretion signal peptide” as used herein refers to a sequence present in the N-terminus of a precursor polypeptide (a pre-peptide or pre-pro-peptide) that directs its translocation across a membrane. Typically, a precursor polypeptide is processed by cleavage of the signal sequence to generate a mature peptide or a pro-peptide. If the product of off-cleavage of the signal peptide is a pro-peptide, the mature peptide is the product of subsequent post-translational modifications that involve further removal of amino acids.

The “S. cerevisiae ura3 gene” refers to the NCBI data base retrievable sequence K02207, which represents the regulatory sequences and gene encoding OMP decarboxylase.

A “stabilizing element” when used herein refers to an element (such as stb which is localised to chromosome III of S. pombe). stb was obtained as a 1295 bp EcoRI fragment from the pFL20 vector (Heyer et al., 1986. Mol. Cell. Biol. 6: 80-89). stb, like other stabilising elements, has the effect of facilitating symmetric segregation of plasmids in connection with mitotic and meiotic cell division, hence ensuring a homogenous population of transformed cells over several generations.

The term “hydrophobic core region” refers to a hydrophobic core in the middle of the signal peptide, i.e. a sequence of amino acids that has an overall hydrophobic hydrophilicity index. The length of such a hydrophobic core region is typically in the range from about 5 to about 20 amino acids in length.

The term “signal peptidase recognition site” refers to a polar region at the C-terminus comprising small, neutral amino acids at positions -1 and -3, i.e. the amino acids alanine, isoleucine, leucine, methionine, valine, glycine, serine, and more rarely proline and threonine. By small amino acids is generally meant amino acids having a side chain containing at most 4 carbon atoms.

“Transformation”: A process by which the genetic material carried by an individual cell is altered by incorporation of exogenous DNA into its genome, either by incorporation of the exogenous DNA into the chromosomal DNA or by introduction of plasmid DNA containing the exogenous DNA.

“Post-translational modification” as used herein refers to the modifications ranging from amino acid changes through to the addition of macromolecule moieties: lipid, carbohydrate, or protein. Many variants of the common amino acids can occur, which can affect the structure or function of the protein. The major class of modifications, however, is represented by glycosylation, N-linked, O-linked, or glycosylphosphatidylinositol(GPI)-linked. Such modifications have roles in protein stability and folding, targeting, and recognition. Glycosylated proteins can be found in all cellular compartments of eukaryotic cells and, intracellularly, O-GlcNAc modification is commonplace. Lipid modification of proteins (acylation, prenylation, GPI-anchoring) is also common, resulting in membrane association, and can play an important role in cell signalling. Targeting and turnover of proteins can also be mediated via covalent protein addition, for example by members of the ubiquitin family. Finally, limited proteolysis as a post-translational modification is also included. It will be understood that post-translational modifications can occur in the host cell of the expression system (as part of the expression process) as well as in vitro. Both possibilities are thus covered by the present use of the term.

### Preferred Embodiments of the Invention

### Chimeric Polynucleotides of the Invention

The first part of the chimeric polynucleotide of the invention is constituted by a nucleotide sequence, which is derived from the S. pombe CPY signal peptide encoding sequence that has the base sequence

### ATGTTAATGAAACAAACCTTCTTGTACTTTTTGCTCACTTGCGTCGTATCCGCT (SEQ ID NO: 1)

that encodes the S. pombe CPY signal peptide consisting of the amino acid sequence MLMKQTFLYFLLTCVVSA (SEQ ID NO: 2).

In general, signal peptides are constituted by 3 distinct regions so that a “functional secretion signal peptide derived from S. pombe CPY” can be described as containing 3 regions: An N-terminal, positively charged region, a central hydrophobic core region, and a C-terminal region that comprises a signal peptidase recognition site.

In most embodiments, the chimeric polynucleotide is free of other targeting sequence encoding nucleotide sequences, since the main purpose of the present invention is to provide for secretion from the host cells into the culture medium of the end product. However, it cannot be excluded that it will sometimes be desirable to include translocation signals that will direct localisation of the polypeptide to specific subcellular locales.

It is preferred that the chimeric polynucleotide of the invention is one encoding a functional signal peptide wherein the overall charge of the N-terminal 6 amino acids is positive. This means that there is some room for substitution, addition and deletion if these operations are performed to preserve the overall charge of the 6 N-terminal amino acids.

Likewise, it is preferred that the encoded hydrophobic core region has a central portion that adopts an alpha-helical conformation in a hydrophobic environment (such as in a membrane's lipid bilayer). Again, this leaves room for conservative substitutions in the CPY signal peptide sequence but it also leaves room for both deletion and addition—the core region is, just prior to off-cleavage of the signal peptide, positioned in the lipid bilayer of the ER, but it seems that the length of this particular region is non-essential for the functionality of the signal peptide, as long as the core region can span the lipid bilayer.

It is also preferred that the C-terminus of the functional secretion signal peptide encoded by the chimeric polynucleotide comprises a signal peptidase recognition site wherein the signal cleavage region is three to six amino acid residues long, with small amino acids in the -1 and -3 positions.

Based on the S. pombe CPY signal peptide, especially preferred chimeric polynucleotides of the invention are those wherein the encoded functional secretion signal peptide is selected from the group consisting of

a) the amino acid sequence MLMKQTFLYFLLTCVVSA (SEQ ID NO: 2),
b) the amino acid sequence of a) wherein at most 6 amino acids have been substituted (typically by substituting conservatively so as not to alter charge, hydrophilicity and conformation of the core region, N-terminal charge etc.),
c) the amino acid sequence of a) or b), wherein at most 4 amino acids have been deleted (this will normally only entail deletions in the N-terminus and in the core region, since the C-terminal signal peptidase recognition site is vulnerable to changes in the amino acid sequence), and
d) the amino acid sequence of a), b) or c), wherein at most 12 amino acids have been added (these will typically be added in the N-terminus or in the hydrophobic core region, especially the latter since the precise length of the hydrophobic core region is not believed to be essential for the functionality of the encoded signal peptide).

The amino acid sequence encoded by the second part of the chimeric polynucleotide is typically a polypeptide product of interest although this does not exclude that the second part encodes short peptides.

For instance, the encoded polypeptide product can be selected from the group consisting of an industrial enzyme; a pharmaceutically active polypeptide such as a hormone, a cytokine, an immunogen, a receptor, a chaperone, an immunoglobulin, an enzyme, and a growth factor; polypeptide food additives; a fluorescent protein such as GFP; transporter proteins such as flavodoxins, globins, metallothioneins, and ABC transporters; toxins; structural proteins such as Kinesin and Tau; inhibitors such as protease inhibitors; and DNA or RNA associated proteins such as domains, homeobox, HMG, PAX, histones, DNA repair, P53, RecA, and ribosomal proteins, but any polypeptide product that it may be desirable to express and secrete in vitro is a putative expression product encoded by the second part of the chimeric polynucleotide. Notably, these all of these polypeptides may be in the form of their respective pro-peptides but can of cause also be mature polypeptides. The option of using the pro-peptide form of a polypeptide or protein product is especially relevant when the mature polypeptide has a biological activity it is desired to control until the time of cleaving of the pro-part of the pro-peptide.

Hence, a particularly preferred chimeric polynucleotide of the invention is constituted by a nucleic acid sequence where the 5′ codon encodes the N-terminal amino acid in the functional secretion signal peptide and where the 3′ codon is a stop codon that follows directly after a codon encoding the C-terminal amino acid in the polypeptide product discussed above. In the most preferred embodiments of the present invention, the first part of the chimeric polynucleotide is constituted by a nucleic acid sequence that encodes SEQ ID NO: 2, and in this context the nucleic acid sequence SEQ ID NO: 1 is the single most preferred embodiment of the first part of the chimeric polynucleotide.

### Vectors and Transformed Host Cells of the Invention

The chimeric polynucleotides of the present invention are suitable as constituents of the expression cassette in expression vectors but also as parts of cloning vectors. As detailed above, there is a serious lack of expression vectors for use in S. pombe and hence also a serious lack of vectors that will be useful in both S. pombe and cells of animal (e.g. insect or mammalian) origin. The presently described vectors fulfil this need.

Hence, another aspect of the invention relates to a vector that comprises the chimeric polynucleotide of the invention as described above. Such a vector is typically a plasmid, a phage, a cosmid, a mini-chromosome, or a virus. The preferred form of the vector is a plasmid.

When the vector of the invention is an expression vector, it may contain a promoter region of yeast origin, especially of S. pombe origin, but in some embodiments it is preferred that it contains a promoter region effective in an animal (e.g. a mammalian virus promoter such as the promoters from the SV40 and CMV genes or a true mammalian promoter such as the promoter from the human chorionic gonadotropin gene) operably linked to the chimeric polynucleotide. The rationale behind the use of such “mammalian purpose” promoters is that the expression construct will be effective in both e.g. S. pombe and in various mammalian host cells, thus minimizing the need for different expression vectors for use in different cell types. It should be noted that experiments performed in Applicant's lab has shown that various mammalian promoters are effective when the host cell for the expression vector is S. pombe.

It is preferred that the vector of the invention comprises a gene encoding at least one selectable marker in order to isolate transformed host cells—such a selectable marker will be chosen according to characteristics of the host cell of choice. In this context, when the expression vector is a plasmid it is especially preferred that detection of the encoded selectable marker requires a high copy number of the plasmid expression vector—this preferred embodiment thus ensures that effectively producing host cells will be preferentially isolated. As shown in the examples, one such suitable selectable marker system for expression in S. pombe is expression of the S. cerevisiae ura3 gene under the control of its native promoter.

Further interesting embodiments of the plasmid vectors of the invention are those that exhibit symmetric segregation of the plasmid in the host cell of choice, thus facilitating the provision of stably transformed cells. This can according to the present invention be accomplished by including a stabilizing element in the expression vector.

For instance, the present examples demonstrate that the stabilizing S. pombe stb element can be introduced into an expression vector adapted for use in S. pombe.

The host cells that are transformed with the vectors of the invention are eukaryotic cells, such as fungal, plant or animal cells.

In the event the host cell is of animal origin, it is preferred that the cell is an insect cell or a cell from a vertebrate, such as a mammal (including a human being). In principle, any cell culture of animal origin is workable, whether from vertebrate or invertebrate culture. However, interest has been greatest in vertebrate cells, and propagation of vertebrate in culture (tissue culture) has become a routine procedure in recent years (Tissue Culture, 1973). Examples of such useful host cell lines are VERO and HeLa cells, Chinese hamster ovary (CHO) cell lines, and W138, BHK, COS-7 293, Spodoptera frugiperda (SF) cells (commercially available as complete expression systems from i.a. Protein Sciences, 1000 Research Parkway, Meriden, Conn. 06450, U.S.A. and from Invitrogen), and MDCK cell lines. In the present invention, an especially preferred cell line is S2 available from Invitrogen, PO Box 2312, 9704 CH Groningen, The Netherlands.

Expression vectors for such cells ordinarily include (if necessary and in addition to the chimeric polynucleotide of the invention) an origin of replication, a promoter located in front of the gene to be expressed, along with any necessary ribosome binding sites, RNA splice sites, polyadenylation site, and transcriptional terminator sequences.

For use in mammalian cells, the control functions on the expression vectors are often provided by viral material. For example, commonly used promoters are derived from polyoma, Adenovirus 2, and most frequently Simian Virus 40 (SV40) or cytomegalovirus, CMV. The early and late promoters of SV40 or virus are particularly useful because both are obtained easily from the virus as a fragment, which also contains the SV40 viral origin of replication (Fiers et al., 1978). Smaller or larger SV40 fragments may also be used, provided there is included the approximately 250 bp sequence extending from the HindIII site toward the BglI site located in the viral origin of replication. Also the immediate early promoter from CMV is of interest. Further, it is also possible, and often desirable, to utilize promoter or control sequences normally associated with the desired gene sequence, provided such control sequences are compatible with the host cell systems.

It has also been demonstrated recently in Applicant's laboratory that the promoter region from the human chorionic gonadotropin gene is effective in the present context (and also in S. pombe).

An origin of replication may be provided either by construction of the vector to include an exogenous origin, such as may be derived from SV40 or other viral (e.g., Polyoma, Adeno, VSV, BPV) or may be provided by the host cell chromosomal replication mechanism. If the vector is integrated into the host cell chromosome, the latter is often sufficient.

However, preferred host cells of the invention are fungal cells, and most preferred are S. pombe cells.

The vectors suitable for transformation of yeast are well known in the art. For S. pombe transformation, vectors having the general characteristics of the vectors shown in the accompanying figures are suitable: A promoter and a transcriptional terminator functional in S. pombe such as those elements derived from the S. pombe nmt1 gene, a gene under the control of the promoter, where the gene includes the coding region for the effective secretion signal peptide discussed herein, an autonomously replicating sequence such as ars1; a selectable marker gene; and a bacterial origin of replication and a selectable marker gene useful in bacteria.

The most preferred cells of the invention are the cells listed above that are stably transformed with an expression vector of the invention.

### Methods of the Invention for Protein Expression

Another aspect of the present invention relates to expression and recovery of polypeptides from host cells.

As detailed above, this aspect of the invention generally utilises the finding that the S. pombe CPY signal peptide having SEQ ID NO: 2 provides for improved yields when expressing secreted polypeptides. The present inventor has recently demonstrated that this improved yield (over e.g. construct utilising the P3 signal peptide) is a consequence of improved secretion and not merely of higher levels of transcribed mRNA—there was no observable difference in the mRNA levels in S. pombe strains transformed with the 2 types of expression vectors, whereas the yields obtained when using the constructs with the CPY signal peptide were 2-3 times higher than those obtained when using the P3 signal peptide.

As mentioned above, step a) of the method of the invention comprises transformation of a host cell with an expression vector that includes a promoter, a coding sequence operably linked thereto, and, optionally, a terminator, wherein said coding sequence comprises a first part encoding a functional secretion signal peptide derived from S. pombe CPY and a second part encoding the polypeptide, said second part being located C-terminally relative to the functional secretion signal peptide, subsequent culture of the transformed host cells under conditions that facilitate expression of the coding sequence and translocation of the polypeptide whereby the functional secretion signal peptide is cleaved from its linkage to the polypeptide, and finally recovery and optionally purification of the polypeptide from the culture.

Methods for transformation will vary according to the choice of host cell, but typically transfection by means of lithium acetate (Okazaki et al. Nucleic acid Res. 18: 6485-6489, 1990, incorporate by reference herein) or electroporation is used in yeast, whereas both transfection and transduction (i.e. transfer of genetic material by means of a viral vector) may be used in cells from multicellular organisms. Moreno et al. 1991, Methods Enzym. 194: 795-823 provides several useful methods for transformation and culture of S. pombe.

More general teachings on transformation and culture of transformed cells (yeast or higher) can be found in Sambrook 3 et al., “Molecular Cloning: A laboratory Manual”, 3^{rd} edition.

The teachings provided above concerning choice of promoter, functional secretion signal peptides, choice of format of vectors, choice of host cells, use of selectable markers, and use of stabilizing elements, apply mutatis mutandis to the method of the invention. The only difference in these teachings and the teachings pertaining to the method of the invention is the precise composition of the coding sequence in the vector, since the method of the invention does not rely on the presence of a chimeric polynucleotide as defined herein. It is however, preferred that the expression vector used in the method of the invention is an expression vector of the invention.

It some embodiments, the method of the invention comprises the further step of subjecting the polypeptide obtained in step (c) to post-translational modification—this entails both post-translational modifications that are effected by the host cell (and subsequently made before recovery of the polypeptide) and modifications made in vitro after recovery of the polypeptide.

Step (a) of the method of the invention normally comprises the steps of introducing the vector into the host cell and subsequently selecting transformants that express a selectable marker gene present in the vector. Useful selectable marker genes have been detailed above.

The invention will be illustrated by means of the following non-limiting examples.

### EXAMPLE 1

### Transformation of Yeast Strains Eg328 and Eg660

S. pombe strains Eg328, h⁹⁰ smt-0 ura4-D18, (ATCC 90720; Styrkarsdottir U et al. Curr. Genet. 23: 184-186, 1993) and Eg660, h+ura4-D18 leu1, (Petersen J et al., 1995. Mol. Cell. Biol. 15: 3697-3707) were transformed with secretory expression vectors according to Okazaki et al. (Nucleic acid Res. 18: 6485-6489, 1990). In brief, 50 ml of culture was grown at 29° C. to a density of 1-2×10⁷ cells/ml in EMM minimal medium (Moreno et al., Methods Enzym. 194: 795-823, 1991) supplemented with 10 mM of L-uridine and/or L-leucine. Cells were collected by centrifuging, washed with sterile water, resuspended in 1 ml of 0.1 M Lithium acetate, pH 4.9 and incubated at 29° C. for 60 minutes, successively. 100 μl of the cell suspension was mixed with 1 μg of the expression vector and 290 μl of 50% PEG4000 and incubated further at 29° C. for 50 minutes. Subsequently, the suspension was incubated at 42° C. for 12 minutes and aliquots were plated on EMM minimal agar medium. Transformants appeared after 3-5 days of incubation at 29° C.

### EXAMPLE 2

### Cultivation of Yeast

The transformed S. pombe strains, Eg328 and Eg660, were grown in EMM+2 mM thiamine+/−10 mM L-leucine to a cell density of 2×10⁵ cells/ml. To induce expression, cells were collected by centrifuging, washed with sterile water, and resuspended in fresh EMM medium without thiamine and incubated further at 29° C. in a rotary shaker. Samples were withdrawn from the cultures 24, 48 and 72 hours after start of induction. Samples were centrifuged briefly to collect cells and the supernatant were analysed directly for secreted products by SDS PAGE and Western analysis.

### EXAMPLE 3

### Identification of the CPY Signal Peptide

Using the S. pombe genome database at the Sanger Institute (www.sanger.ac.uk/Projects/S_pombe) a multitude of putative ER translocated proteins were identified. These proteins were analysed for putative secretion signal peptides using the SignalP software (Nielsen H and Krogh A 1997, Protein Engineering 10:1-6; www.cbs.dtu.dk), and among the positives was the cpy1 gene or SPAC19G12.11C (Tabuchi M et al. 1997, J. Bacteriol. 179: 4179-4189; www.sanger.ac.uk/Projects/S_pombe). The cleavage site for the signal peptidase was predicted to be between amino acids 18 and 19 in the cpy1 gene product (probability>88%).

The CPY signal peptide is encoded by the following DNA sequence:

### ATGTTAATGAAACAAACCTTCTTGTACTTTTTGCTCACTTGCGTCGTATCCGCT (SEQ ID NO: 1).

The amino acid sequence of the CPY signal peptide:

### MLMKQTFLYFLLTCVVSA (SEQ ID NO: 2).

### EXAMPLE 4

### Preparation of General-Purpose Secretory Expression Vectors

The secretory expression vectors described below are all derived from pSFL172 (Forsburg S L and Sherman D A 1997, Gene 191(2): 191-195; ATCC 87609).

Generation of a general-purpose secretory vector harbouring the S. pombe CPY signal peptide was prepared as described in the following. The CPY signal peptide was linked to green fluorescent AFP (Qbiogene) and a Kozak element by PCR using PQBI 25-fA1 (Qbiogene) as template and the oligonucleotides:

(SEQ ID NO: 3)

5′-TAACTCGAGACCATGTTAATGAAACAAACCTTCTTGTACTTTTTGCT
CACTTGCGTCGTATCCGCCATGGCTAGCAAAGGAGAAGAACTCTT
and

(SEQ ID NO: 4)

5′-TAAGTCGACGCGGCCGCGCTAGCGGATCCTCAATCGATGTTGTACAG
TTCATCCA

as primers.

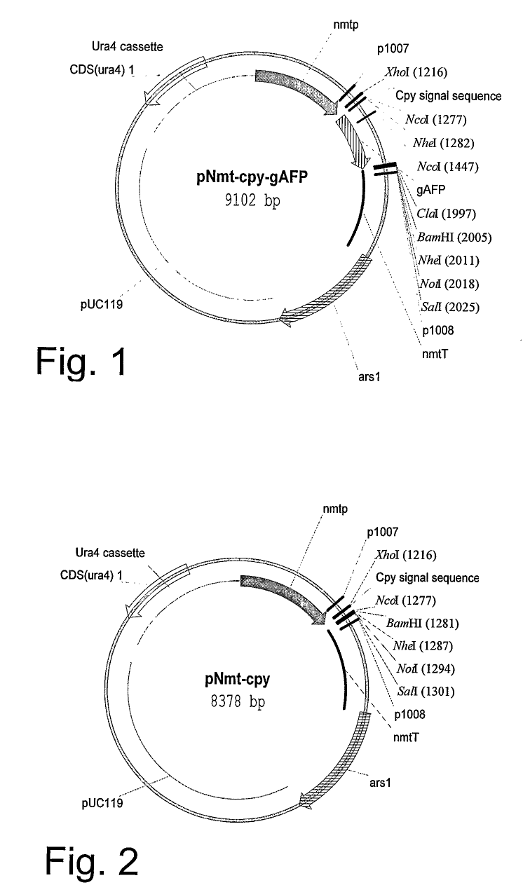

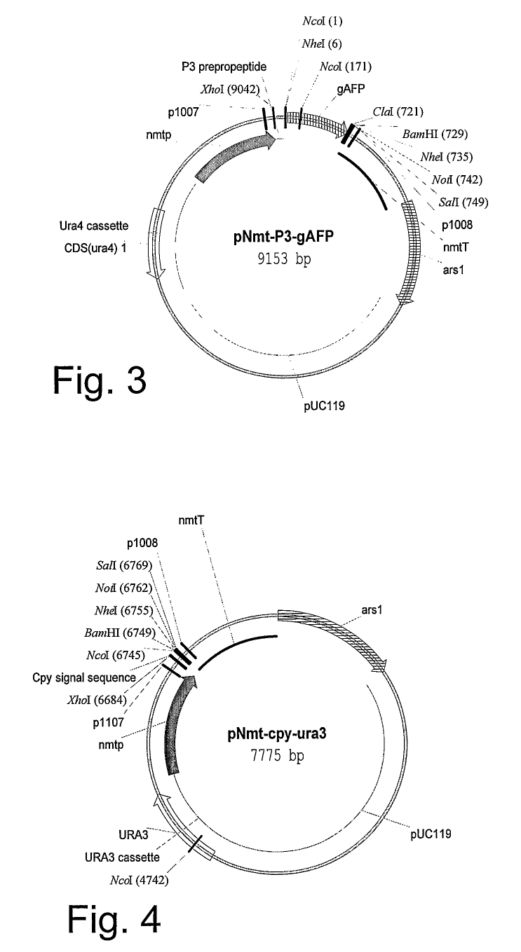

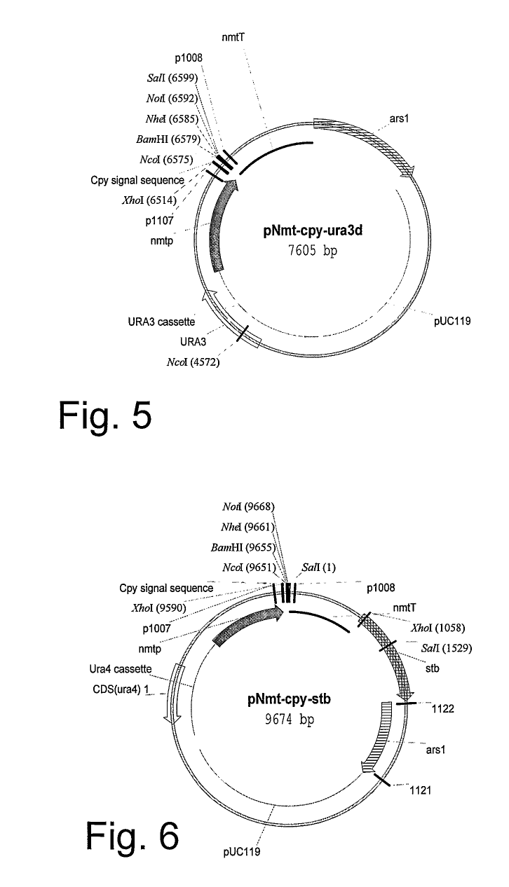

The generated PCR product was restricted with XhoI and SalI and ligated into XhoI/salI digested and dephosphorylated pSFL172, giving pNmt-cpy-gAFP (FIG. 1). pNmt-cpy-gAFP was restricted with NcoI+BamHI, thereby deleting AFP, end-filled with T4 DNA polymerase and religated. The resulting general-purpose secretory vector, pNMT-cpy (FIG. 2), contains the following features: five unique restriction sites, NcoI, BamHI, NheI, NotI and SalI, for cloning in-frame with the CPY secretion signal peptide; Regulated expression from the strong nmt1 promoter; nmt1 trailer for transcriptional termination and poly-adenylation; Ars1 and pMB1/f1 elements for autonomous replication in S. pombe and E. coli, respectively; ura4 and β-lactamase genes function as selection markers in S. pombe and E. coli, respectively.

A secretory vector containing the P3 pre-pro-peptide (WO 96/23890), derived from the S. pombe P-factor precursor, was constructed as described in the following. First, the P3 pre-pro-peptide was synthesized by PCR using genomic DNA from Eg328 (ATCC 90720) as template and the oligonucleotides:

(SEQ ID NO: 5)

5′- TCTCGAGAACATGAAGATCACCGCTGTCATT
and

(SEQ ID NO: 6)

5′- TCCATGGCACGCTTCTTAAGGCTAACTGAAACCACACCAGGA

as primers.

Secondly, green fluorescent AFP (Qbiogene) was amplified by PCR using pQBI 25-fA1 (Qbiogene) as template and the oligonucleotides:

(SEQ ID NO: 7)

5′- TAACCATGGCTAGCAAAGGAGAAGAACTCTT
and

(SEQ ID NO: 8)

5′- TAAGTCGACGCGGCCGCGCTAGCGGATCCTCAATCGATGTTGTACA
GTTCATCCA.

Thirdly, the PCR products comprising the P3 pre-pro-peptide and AFP were restricted with XhoI+NcoI and NcoI and SalI, respectively, and ligated into XhoI/SalI digested and dephosphorylated pSFL172. The resulting P3 secretory expression vector, pNmt-P3-gAFP (FIG. 3), is identical to pNmt-cpy-gAFP, apart from the secretion signal peptides.

Preliminary results indicate that the described CPY signal peptide confers efficient secretion to recombinant proteins.

Further, and very important, the results indicate that the CPY signal peptide is 2-3 fold more efficient in recombinant protein secretion than the P3 signal peptide, which is described in the literature to be very effective.

Finally, the cleavage point of the signal peptidase was confirmed to be between amino acids 18 and 19 in the CPY precursor protein, giving a well defined and homogenous secreted protein product.

### EXAMPLE 5

### Preparation of Secretory Expression Vectors with Increased Copy Number

In order to increase the copy number of the general-purpose secretory expression vector, the S. pombe ura4 marker gene was replaced with the ura3 gene from S. cerevisiae. The ura3 gene only weakly complements an ura4 null mutation in S. pombe. The ura3 gene was amplified by PCR using pFL20 (Heyer et al. 1986, Mol. Cell. Biol. 6: 80-89) as template and the oligonucleotides:

(SEQ ID NO: 9)

5′- GTCCATAAAGCTTTTCAATTCATCT
and

(SEQ ID NO: 10)

5′- TCTGCAGAGCTTTTTCTTTCCAATTTTTTTT

as primers.

The resulting PCR product was restricted with HinDIII+PstI and ligated into HinDII11 PstI digested pNmt-cpy, giving pNmt-cpy-ura3 (FIG. 4).

To further increase the copy number of the pNmt-cpy-ura3 vector the promoter of the ura3 marker gene was modified as described in the following. All sequences localized upstream to -45 bp with respect to the initiation ATG was deleted by PCR using pFL20 as template and the oligonucleotides:

(SEQ ID NO: 11)

5′- TAAGCTTGGTACCCAACTGCACAGAACAAAAATTGCAGGAAACGAA
GATAAATCA

(SEQ ID NO: 12)

5′- TCTGCAGAGCTTTTTCTTTCCAATTTTTTTT

as primers.

The resulting PCR product was restricted with HinDIII+PstI and ligated into HinDII11 PstI digested pNmt-cpy, giving pNmt-cpy-ura3d (FIG. 5).

### EXAMPLE 6

### Preparation of Secretory Expression Vector with Increased Stability

In order to improve the symmetric segregation of the general-purpose secretory expression vector, a so-called stb element from S. pombe was inserted in pNmt-cpy as described in the following.

The stb element, localised to chromosome III was obtained as a 1295 bp EcoRI fragment from the pFL20 vector (Heyer et al. 1986, Mol. Cell. Biol. 6: 80-89). The EcoRI fragment comprising stb was ligated into EcoRI digested and dephosphorylated pNmt-cpy, giving pNmt-cpy-stb (FIG. 6).

1. A chimeric polynucleotide that comprises
a)
a first part encoding a functional secretion signal peptide derived from S. pombe carboxypeptidase Y (CPY), and
a second part linked directly to the 3′ end of the first portion,
wherein the second part encodes an amino acid sequence that is not naturally associated with an S. pombe carboxypeptidase Y signal peptide and which does not include amino acids 1-5 of the S. pombe carboxypeptidase Y pro-peptide, or
b) a nucleotide sequence complementary to the nucleotide sequence in a).

2. The chimeric polynucleotide according to claim 1, wherein the overall charge of the N-terminal 6 amino acids of the functional secretion signal peptide is positive.

3. The chimeric polynucleotide according to claim 1, wherein the functional secretion signal peptide comprises a hydrophobic core region wherein the central portion adopts an alpha-helical conformation in a hydrophobic environment.

4. The chimeric polynucleotide according to claim 1, wherein the C-terminus of the functional secretion signal peptide comprises a signal peptidase recognition site wherein the signal cleavage region is three to six amino acid residues long, with small amino acids in the -1 and -3 positions.

5. The chimeric polynucleotide according to claim 1, wherein the functional secretion signal peptide is selected from the group consisting of
a) the amino acid sequence MLMKQTFLYFLLTCVVSA (SEQ ID NO: 2),
b) the amino acid sequence of a) wherein at most 6 amino acids have been substituted,
c) the amino acid sequence of a) or b), wherein at most 4 amino acids have been deleted, and
d) the amino acid sequence of a), b) or c), wherein at most 12 amino acids have been added.

6. The chimeric polynucleotide according to claim 1, wherein the second part encodes a polypeptide product.

7. The chimeric polynucleotide according to claim 6, wherein the polypeptide product is selected from the group consisting of an industrial enzyme; a pharmaceutically active polypeptide such as a hormone, a cytokine, an immunogen, a receptor, a chaperone, an immunoglobulin, an enzyme, and a growth factor; polypeptide food additives; a fluorescent protein such as GFP; transporter proteins such as Flavodoxins, Globins, Metallothioneins, and ABC transporters; toxins; structural proteins such as Kinesin and Tau; inhibitors such as Protease inhibitors; and DNA or RNA associated proteins such as Domains, Homeobox, HMG, PAX, Histones, DNA repair, P53, RecA, and ribosomal proteins.

8. The chimeric polynucleotide according to claim 1, where the 5′ codon encodes the N-terminal amino acid in the functional secretion signal peptide and where the 3′ codon is a stop codon that follows directly after a codon encoding the C-terminal amino acid in a polypeptide product.

9. A vector comprising the chimeric polynucleotide according to claim 1.

10. The vector according to claim 9, which is selected from the group consisting of a plasmid, a phage, a cosmid, a mini-chromosome, and a virus.

11. The vector according to claim 9, which is a cloning vector.

12. The vector according to claim 9, which is an expression vector.

13. The vector according to claim 12, which has an animal virus derived promoter region or an animal promoter region operably linked to the chimeric polynucleotide.

14. The vector according to claim 12, which comprises a selectable marker gene.

15. The vector according to claim 14, wherein detection of the selectable marker requires a high copy number of the expression vector.

16. The vector according to claim 15, wherein the selectable marker is expression of the S. cerevisiae ura3 gene under the control of its native promoter.

17. The vector according to claim 9, which further comprises a stabilizing element that improves symmetric segregation of a plasmid.

18. The vector according to claim 17, wherein the stabilizing element is the S. pombe stb element.

19. A host cell transformed with the vector according to claim 9.

20. The host cell according to claim 19, which is a eukaryotic cell, such as a fungal, plant or animal cell.

21. The host cell according to claim 20, where the animal cell is an insect cell or a cell from a vertebrate, such as a mammal.

22. The host cell according to claim 20, wherein the fungal cell is a S. pombe cell.

23. A method for recombinant preparation of a polypeptide, comprising
a) transforming a host cell with an expression vector that includes a promoter, a coding sequence operably linked thereto, and, optionally, a terminator,
wherein the coding sequence comprises a first part encoding a functional secretion signal peptide derived from S. pombe CPY and a second part encoding the polypeptide that is located C-terminally relative to the functional secretion signal peptide,
b) culturing the transformed host cells under conditions that facilitate expression of the coding sequence and translocation of the polypeptide whereby the functional secretion signal peptide is cleaved from its linkage to the polypeptide, and
c) recovering and optionally purifying the polypeptide from the culture.

24. The method according to claim 23, which comprises the further step of subjecting the polypeptide obtained in step c to post-translational modification.

25. The method according to claim 23, wherein step (a) comprises the steps of introducing the vector into the host cell and subsequently selecting transformants that express a selectable marker gene present in the vector.

26. The method according to claim 23, wherein the vector is selected from the group consisting of a plasmid, a phage, a cosmid, a mini-chromosome, and a virus.

27. The method according to claim 23, wherein the vector has an animal virus derived promoter region operably linked to the chimeric polynucleotide.

28. The method according to claim 23, wherein the vector comprises a selectable marker.

29. The method according to claim 28, wherein detection of the selectable marker requires a high copy number of the expression vector.

30. The method according to claim 29, wherein the selectable marker is expression of the S. cerevisiae URA3 gene under the control of its native promoter.

31. The method according to claim 23, wherein the vector comprises a stabilizing element that improves symmetric segregation of a plasmid in a host cell.

32. The method according to claim 31, wherein the stabilizing element is the S. pombe stb element.

33. The method according to claim 23, wherein the expression vector is the expression vector according to claim 12.

34. The method according to claim 23, wherein the host cell is a eukaryotic cell, such as a fungal, plant or animal cell.

35. The method according to claim 34, where the animal cell is an insect cell or a cell from a vertebrate, such as a mammal.

36. The method according to claim 34, wherein the fungal cell is a S. pombe cell.

37-40. (canceled)
